(19) United States
(12) Patent Application Publication
(10) Pub. No.: US 2012/0295353 A1
(43) Pub. Date: Nov. 22, 2012

(54) METHODS OF MAKING AND USING POLYMERS AND COMPOSITIONS

(76) Inventors: Yulong Hong, Painted Post, NY (US); Thomas Mark Leslie, Horseheads, NY (US); Hui Su, Big Flats, NY (US); Jian Tan, Ithaca, NY (US)

(21) Appl. No.: 13/470,536

(22) Filed: May 14, 2012

Related U.S. Application Data
(60) Provisional application No. 61/488,420, filed on May 20, 2011.

(51) Int. Cl. C12N 5/071; B29C 35/08; B29C 41/00; B05D 7/00; B05D 3/06

(52) U.S. Cl. 435/402; 427/517; 264/496

(57) ABSTRACT

Disclosed are methods of making and using polymers compositions. The polymer compositions may have monomer/oligomer mixtures that may have at least one silicone monomer or oligomer and at least one non-silicone monomer or oligomer, at least one crosslinker, and/or at least one polymerization initiator. The polymer compositions are cured, after which they may be useful in bioapplications, such as for use as freestanding films or coatings on a substrate, such as a mold, for cell culture.

### CROSS-REFERENCE TO RELATED APPLICATIONS

This application claims the benefit of priority under 35 U.S.C. §119 of U.S. Provisional Application Ser. No. 61/488,420 filed on May 20, 2011, the content of which is relied upon and incorporated herein by reference in its entirety.

### TECHNICAL FIELD

The disclosure relates to methods of making and using polymers comprising at least one silicone monomer or oligomer and at least one non-silicone monomer or oligomer. Methods of making polymers according to various embodiments of the disclosure comprise mixing at least one silicone monomer or oligomer with at least one non-silicone monomer or oligomer to form a monomer/oligomer mixture, and curing the monomer/oligomer mixture. Additional components may also be added to the monomer/oligomer mixture, such as, for example, at least one crosslinker and/or at least one polymerization initiator, to form a polymer composition. The polymer compositions may then be cured.

Methods of using the polymers and compositions may comprise, for example, coating a substrate with a polymer composition described herein. In various embodiments, the methods may further comprise curing the polymer composition on the substrate to form a cured polymer composition coating on the substrate, or forming a freestanding film comprising the cured polymer compositions. Such films and coated substrates may be useful in bioapplications, such as, for example, in cell culture applications.

### BACKGROUND

Cell culture is a process by which cells are grown in vitro under artificial conditions. Several different factors can affect the cell culture process and cellular function. One factor of interest is the interaction of the cell with its surrounding environment. The extracellular matrix (ECM), which is the extracellular part of a cell that provides structural support to the cell, is essential for cell survival and function, providing a dynamic chemical environment for cellular activities. The ECM is mechanically soft, multi-dimensional, and permeable, permitting the exchange of nutrients and gases such as oxygen.

In the laboratory, most cells are cultured on a substrate. Current cell culture substrates, however, present challenges for those trying to achieve optimal cell growth conditions. For example, one common type of cell culture substrate is a polystyrene-based culture surface, which is made of flat and rigid plastics having poor gas permeability. Such simplified surfaces are very different from the complex in vivo conditions that consist of, for example, soluble growth factors, insoluble ECM components, and neighboring cell membranes.

In addition, artificial conditions, such as those found with current substrates, may lead to cell behavior that does not accurately reflect true physiological activity. For example, primary cells may lose their differentiation and phenotype under such conditions. Furthermore, studies have shown that normal cells can turn into cancer cells on rigid substrates. As such, time and effort has been put into finding synthetic materials that possess properties needed to support cellular activities in an environment similar to the ECM.

Polydimethylsiloxane (PDMS) has been identified as a promising material with such desirable attributes. PDMS is soft, oxygen permeable, and optically transparent, and has shown great potential as a cell culture substrate. However, PDMS has a disadvantage of being extremely hydrophobic and difficult to modify chemically. It takes up hydrophobic drug molecules irreversibly from the culture medium, thereby making it difficult to use for drug function screening. It can also present batch-to-batch and lot-to-lot variations since it is a complex, two part curing mixture.

Accordingly, there is a need for synthetic materials that may, in at least certain embodiments, not have some or all of the disadvantages associated with PDMS.

### SUMMARY

According to various embodiments of the disclosure are described methods of making polymers comprising at least one silicone monomer or oligomer and at least one non-silicone monomer or oligomer, in the form of a monomer/oligomer mixture. With reference to the at least one silicone monomer or oligomer, and the at least one non-silicone monomer or oligomer, the term “monomer/oligomer mixture” is intended to include a mixture of monomers, a mixture of oligomers, and a mixture of monomers and oligomers. The methods further comprise, in various embodiments, adding at least one crosslinker and/or polymerization initiator to the monomer/oligomer mixture. In further embodiments, the mixture is cured. For example, the mixture may be cured on a substrate, such as a cell culture substrate. In further embodiments, the cured mixture may remain under an actinic heat source, such as a LED lamp, beyond its curing time without deleterious. The mixture may, in some embodiments, form a coating of a cured polymer composition on a substrate. The mixture may also form a film comprising a cured polymer composition that in some embodiments may be peeled off a support to form a freestanding film. The support may be any shaped surface or structure able to sustain the mixture until the film is peeled off.

The polymer compositions disclosed herein may be useful, at least in certain embodiments, in bioapplications. For example, the cured polymer compositions may be useful for cell culture techniques and methods involving highly metabolic cells. Additionally, the polymer compositions disclosed herein may be useful, in various exemplary embodiments, as a coating or a film for use as a cell culture substrate. For example, in various embodiments, the compositions and/or coatings and films may exhibit properties that are useful for cell culture applications, such as some degree of optical transparency. In further embodiments, the compositions and/or coatings and films may exhibit some degree of reduced drug uptake, relative to known compositions, such as, for example, PDMS. In further embodiments, the compositions and/or coatings and films may exhibit some degree of oxygen permeability. In yet further embodiments, the compositions and/or coatings and films may be biocompatible, e.g. may be favorable to cell growth conditions, may be less toxic to cells than known compositions, or may be nontoxic to cells. In further embodiments, the compositions and/or coatings and films may be moldable. In yet further embodiments, the compositions and/or coatings and films may have properties of flexibility and/or low modulus. In still further embodiments, the compositions and/or coatings and films may be used as potential scaffolds in tissue engineering. In various embodiments of the disclosure, the compositions and/or coatings and films may exhibit more than one of the aforementioned properties; however, it should be noted that some or all of the aforementioned properties of the compositions and/or coatings and films may not be present in at least certain exemplary embodiments, yet such embodiments are intended to be within the scope of the disclosure.

Additional objects and advantages of the disclosure are set forth in the following description. Both the foregoing general summary and the following detailed description are exemplary only, and are not intended to be restrictive of the invention as claimed. Further features and variations may be provided in addition to those set forth in the description. For instance, the disclosure is intended to include various combinations and sub-combinations of the features disclosed in the detailed description. In addition, it will be noted that the order of the steps presented need not be performed in that order in order to practice various aspects of the disclosure.

### BRIEF DESCRIPTION OF THE DRAWINGS

The following figures, which are described below and which are incorporated in and constitute a part of the specification, illustrate exemplary embodiments and are not to be considered limiting of the scope of the disclosure.

FIG. 1 is a graphical representation comparing the water contact angle of polymer compositions prepared in accordance with exemplary embodiments of the present disclosure obtained right after the water drop is applied.

FIG. 2 is a graphical representation comparing the change in contact angle as a function of time for different polymer compositions prepared in accordance with exemplary embodiments of the present disclosure.

FIG. 3 is a graphical representation comparing the effect of curing time on monomer release from films of different polymer compositions prepared in accordance with exemplary embodiments of the present disclosure as measured by UV absorption.

FIG. 4A is a graphical representation comparing the effect of curing time of 1 minute and polymer coating thickness on monomer release in different polymer compositions prepared in accordance with exemplary embodiments of the present disclosure.

FIG. 4B is a graphical representation comparing the effect of curing time of 5 minutes and polymer coating thickness on monomer release in different polymer compositions prepared in accordance with exemplary embodiments of the present disclosure.

FIG. 5 is a graphical representation comparing the uptake of the drug nefazadone by polymer compositions prepared in accordance with exemplary embodiments of the present disclosure.

FIG. 6A is a graphical representation comparing the absorption of Tamoxifen, an exemplary hydrophobic drug, by different polymer compositions prepared in accordance with exemplary embodiments of the present disclosure.

FIG. 6B is a graphical representation comparing the absorption of Tamoxifen, an exemplary hydrophobic drug, by PDMS at varying thickness. PS is a polystyrene control.

FIG. 7A is a photomicrograph showing cell growth of a breast cell culture on a commercially available cell culture substrate, Tissue Culture Treated polystyrene (TCT-PS).

FIG. 7B is a photomicrograph showing cell growth of a breast cell culture on a substrate coated by a polymer composition prepared in accordance with an exemplary embodiment of the present disclosure.

FIG. 8A is a graphical representation of a toxicity study with HepG2/C3A cells in cell culture by different polymer compositions prepared in accordance with exemplary embodiments of the present disclosure.

FIG. 8B is a graphical representation of the cell viability and retention study of HepG2/C3A cells in cell culture by different polymer compositions prepared in accordance with exemplary embodiments of the present disclosure.

FIG. 9 is a photomicrograph representation comparing primary human hepatocytes (PHH) on collagen, Matrigel™ overlay (MOL) and synthetic substrates prepared in accordance with an exemplary embodiment of the present disclosure.

FIG. 10 is a graphical representation of the viability of PHH on various substrates prepared according to various embodiments of the disclosure.

FIG. 11 is a graphical representation of the CPY3A4 activity of PHH on various substrates prepared according to various embodiments of this disclosure.

### DESCRIPTION OF EXEMPLARY EMBODIMENTS

As discussed above, the disclosure relates, in various embodiments, to methods of making and using polymers and compositions comprising the same. In at least some exemplary embodiments, the polymers and compositions comprising them may be useful in bioapplications, such as cell culture substrate coatings or freestanding films, and tissue engineering.

In various embodiments, the polymers useful in the methods described herein may be formed from a monomer/oligomer mixture comprising at least one silicone monomer or oligomer and at least one non-silicone monomer or oligomer. The polymers may be in any form, such as, for example, random, block, etc.

The at least one silicone monomer or oligomer may be any silane(meth)acrylate monomer or oligomer that is silicone-based and contains at least one (meth)acrylate moiety. The at least one (meth)acrylate moiety may include different formulations comprising a silicone molecule associated with a (meth)acrylic or (meth)acrylamide group.

In various exemplary embodiments, the (meth)acrylate moiety of the silicone monomer or oligomer may be chosen from monomers or oligomers of Formula 1:

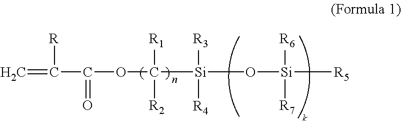

wherein the substituents R, R₁, and R₂ can be H, CH₃ or alkyl, R₃, R₄ and R₅, can be H, CH₃ or alkyl, and n may be from 1 to 10 and k may be from 0 to 25. R₆ and R₇ may be H, CH₃, OSi(R)₃. In other embodiments, the substituents R, R₁, R₂, R₃, R₄, R₅, R₆ and R₇ may be chosen from H, CH₃, OH, phenyl, O—Si(CH₃)₃ or alkyl and cycloalkyl. Still other substituents may be incorporated in other embodiments. By way of example only, monomers or oligomers of Formula 1 may be chosen from (meth)acryloxyethoxy-trimethylsilane (SIM0), also represented by the structure

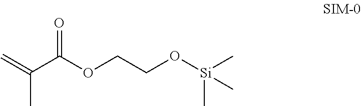

available from Gelest, Inc., of Morrisville, Pa., as well as the monomers or oligomers represented by but not limited to the following structures

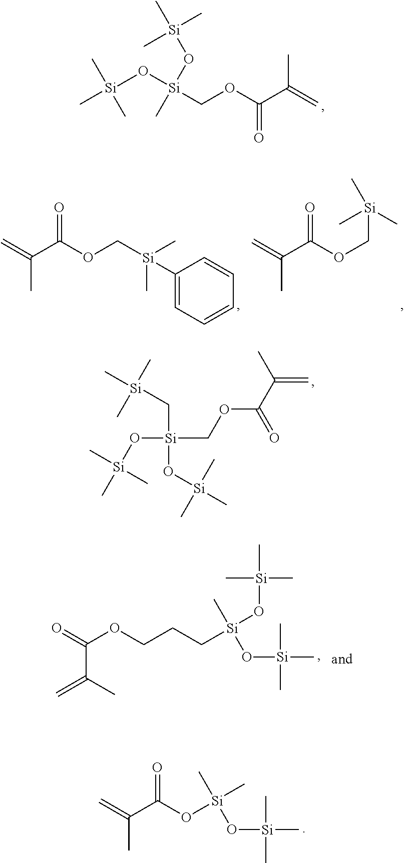

In further exemplary embodiments, the (meth)acrylate moiety of the silicone monomer or oligomer may be chosen from monomers or oligomers of Formula 2:

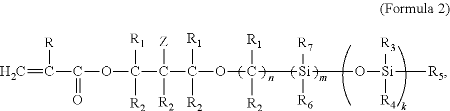

wherein the substituents R, R₁, R₂, R₃, R₄ and R₅ may be chosen from H, CH₃, alkyl, cycloalkyl, phenyl and O—Si(CH₃)₃, and Z can be chosen from H, CH₃, OH, a halide or alkyl, n, m, and k may be from 0 (zero) to 25 but m and k may not be 0 (zero) at the same time. A halide as disclosed herein may include F, Cl, Br or I. Still other substituents may be incorporated in other embodiments. By way of example only, monomers or oligomers of Formula 2 may be chosen from (3-(meth)acryloxy-2-hydroxypropoxy)-propyl-bis(trimethylsiloxy)methylsilane (SIM15), available from Gelest, Inc., also represented by the structure

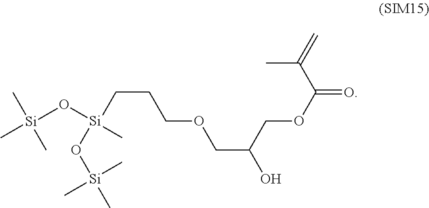

Further exemplary silane(meth)acrylate monomers or oligomers may be chosen from those represented by but not limited to the following structures

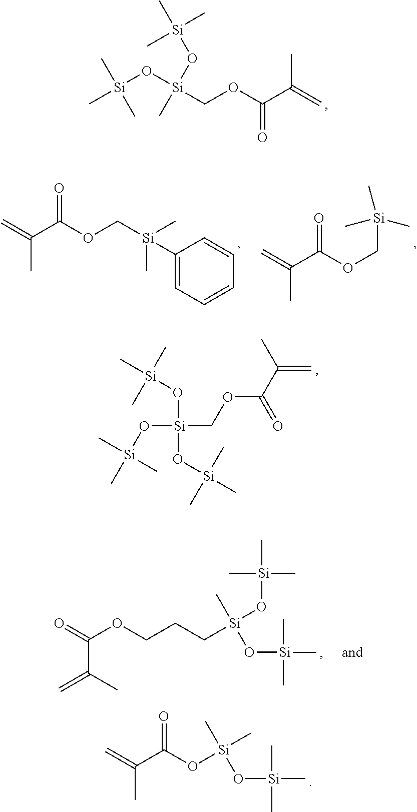

The non-silicone monomer or oligomer may be, for example, a hydrophilic monomer or oligomer or a hydrophobic monomer or oligomer. The non-silicone monomer or oligomer may be chosen based on, for example, compatibility with a particular cell type or the particular intended application. For example, it may be possible, in various embodiments, to either increase or decrease the hydrophobicity/hydrophilicity of the polymer composition by choice and/or amount of the non-silicone monomer or oligomer used.

In various embodiments, the at least one non-silicone monomer or oligomer may be chosen from a hydrophilic monomer or oligomer, such as a hydrogel-forming monomer or oligomer. By way of example only, hydrogel forming-monomers or oligomers may be chosen from acrylamide, (meth)acrylamide,

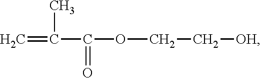

2-hydroxyethyl(meth)acrylate (HEMA),

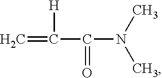

### N,N-dimethylacrylamide (DMA),

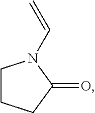

1-vinyl-2-pyrrolidinone (VP), and

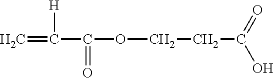

carboxyethyl acrylate (CEA), and the like, and mixtures thereof.

In various exemplary embodiments, the silicone monomer or oligomer can be mixed with the non-silicone monomer or oligomer in any ratio, such as a ratio that ranges from about 100% to about 0%; about 83% to about 17%; about 75% to about 25%; about 67% to about 33%; about 50% to about 50%; about 0% to about 100%; about 17% to about 83%; about 25% to about 75%; or about 33% to about 67%.

In various exemplary embodiments, at least one crosslinker may be added to the monomer/oligomer mixture. For example, the at least one crosslinker can be a monomer or oligomer, such as a hydrophilic monomer or oligomer or a hydrophobic monomer or an oligomer, or a polymer. In certain embodiments, the crosslinker may be chosen from oligomers or polymers of Formula 3:

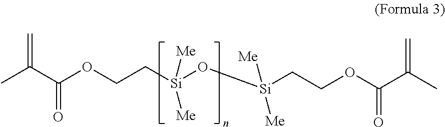

wherein n is a natural number ranging from 0 to 100 or more. By way of example only, crosslinkers of Formula 3 may be chosen from (meth)acryloxypropyl terminated polydimethylsiloxanes, including but not limited to, R31, DMS-R 05; DMS-R11; DMS-R 18; GP 446; GP 478, all available from Gelest, Inc., and represented by the structures

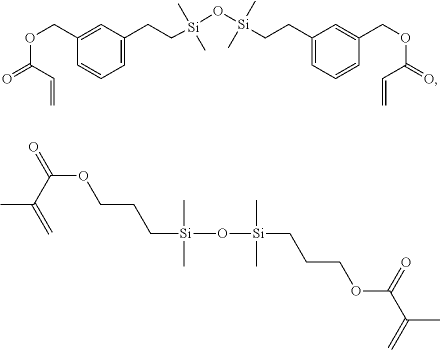

and the like, and mixtures thereof.

In further exemplary embodiments, the crosslinker may be chosen from monomers or oligomers of Formula 4:

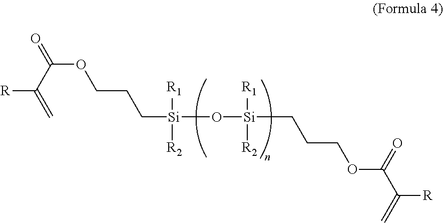

wherein the substituents R, may be chosen from H or CH₃ and R₁, R₂, R₃, and may be chosen from H, CH₃, alkyl, cycloalkyl, phenyl, and O—Si(CH₃)₃ and n=1 to 50. There may be other crosslinkers of this type where n ranges from 1 to about 31. Still other substituents may be incorporated in other embodiments. By way of example only, crosslinkers of Formula 4 may be chosen from 1,3-bis(3-(meth)acryloxypropyl)tetrakis-(trimethylsiloxy)-di-siloxane (SIB), available from Gelest, Inc., represented by the structure

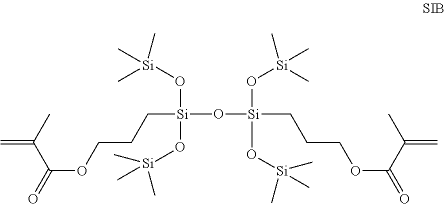

and the like, and mixtures thereof.

In further exemplary embodiments, the crosslinker may be chosen from the following structures

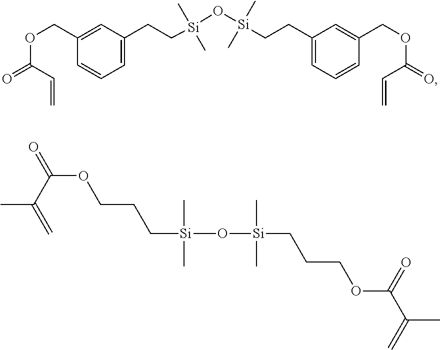

and the like, and mixtures thereof.

In further exemplary embodiments, the crosslinker may be chosen from monomers or oligomers of Formula 5:

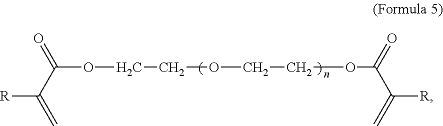

wherein the substituents R and n may be R═CH₃ and n ranges from 0 to 100 or more. Still other substituents may be incorporated in other exemplary embodiments. In further exemplary embodiments, the crosslinker may also be chosen from various acrylates such as hexanediol diacrylate, glycerol di(meth)acrylate, and the like known to those skilled in the art, or mixtures thereof. Additionally, a silicone crosslinker may be used. Additional examples of crosslinkers include divinyl benzene, triallyl isocyanurate, and pentaerytritol tetraacrylate. In yet further exemplary embodiments, various combinations and mixtures of the crosslinkers mentioned herein may be used.

In various exemplary embodiments, the at least one crosslinker can be added to the monomer/oligomer mixture in an amount ranging from about 0.3% to about 100% ratio by weight of crosslinker to monomer/oligomer mixture. By way of example only, the crosslinker may be added to the monomer/oligomer mixture in a ratio of 3% by weight of crosslinker to monomer/oligomer mixture. The degree of crosslinking for any given crosslinker varies and depends on numerous characteristics of the crosslinker, including the structure, number of side branches and the size of the crosslinker, to name a few, each contributing to different properties in the monomer/oligomer mixture.

In various embodiments, at least one polymerization initiator may also be added to the monomer/oligomer mixture. The polymerization initiators may be, for example, photo initiators, thermal initiators, chemical (Red-Ox) or e-beam initiators. A non-limiting example of a useful photo initiator includes, but is not limited to, Irgacure 819. In some embodiments of the disclosure, exposure to e-beam or gamma radiation may cause polymerization without the need of an initiator.

The polymerization initiator may, in various exemplary embodiments, first be dissolved in a solvent. The solvent may be chosen from, for example, one of the monomers, a hydrocarbon or alcohols. By way of example only, the solvent may be chosen from ethanol or various isopropyl isomers.

In various embodiments, the polymerization initiator may be added to the solvent in a concentration of up to about 10%, such as up to about 5%, or up to about 2%. Once the polymerization initiator is dissolved in the solvent, the solution may be added to the monomer/oligomer mixture. The amount of polymerization initiator solution added to the mixture may be chosen such that the amount of polymerization initiator added may range up to about 2% or up to about 5% of the monomer/oligomer mixture by weight, such as up to about 1% by weight, such as about 0.3% to about 0.5% by weight.

The polymer composition comprising a monomer/oligomer mixture, at least one crosslinker and/or at least one polymerization initiator, may be cured by methods known in the art, in order to form a cured polymer composition. In at least certain exemplary embodiments, curing methods may be chosen that provide optimal cell growth conditions. For example, in various embodiments, curing, which may optionally be carried out after forming films or coating substrates with the composition comprising the polymer, may, in at least some embodiments, be carried out at room temperature.

The curing process may, in some embodiments, be carried out by an actinic energy source, a source of electromagnetic radiation that is capable of producing photochemical reactions, and that does not emit long wavelength radiation known to create heat. This may be useful, for example, in applications where a particular type of biomolecule or cell is sensitive to heat. By way of example only, curing may be carried out with wavelength-specific light-emitting diode (LED) UV lamps. In some embodiments, the wavelength is chosen based on the polymerization initiator being used, in order to achieve the desired effectiveness. Any lamp may be used that emits longer wavelengths, such as, for example, a wavelength at least shorter than about 650 nm and at least longer than about 200 nm. Alternatively, LED lamps can be used in certain preferred embodiments with a narrow wavelength distribution over vapor lamps. Any LED lamp may be used that emits longer wavelengths, such as, for example, a wavelength at least shorter than about 650 nm and at least longer than about 300 nm. By way of example, the wavelength may range from about 350 nm to about 550 nm. In certain embodiments, the LED lamp may be chosen from those exhibiting a relatively narrow wavelength distribution, such as about 382±5 nm. In certain other embodiments, an LED lamp with a wavelength of about 365 nm to 415 nm may be used. In at least some embodiments, curing may take place in an inert atmosphere, such as under nitrogen.

Curing time may be chosen by one of skill in the art according to various parameters, such as the components of the composition and the method of curing used. By way of example only, curing time may range from about 30 seconds to about 10 minutes, such as about 5 minutes.

In further exemplary embodiments are disclosed methods of coating a substrate, such as a cell culture substrate, comprising applying a polymer composition comprising a monomer/oligomer mixture comprising at least one silicone monomer or oligomer and at least one non-silicone monomer or oligomer, at least one crosslinker, and/or at least one polymerization initiator, as disclosed herein, to a substrate. In various embodiments, the methods further comprise curing the polymer composition on the substrate by actinic radiation to form a cured polymer composition coating on the substrate.

Further exemplary embodiments relate to coated substrates, wherein the substrate is coated with a polymer composition comprising a monomer/oligomer composition comprising at least one silicone monomer or oligomer and at least one non-silicone monomer or oligomer, at least one crosslinker and/or at least one polymerization initiator, as disclosed herein, wherein the polymer composition is cured by actinic radiation.

In certain embodiments, the monomer/oligomer mixture can be applied to a substrate in an amount ranging from about 0.1 μl to about 1 ml, depending on the size of the surface to be coated. The thickness of the coating on the substrate may range, for example, from about 50 nm to about 300 μm, depending on the size and area of the surface to be coated. In certain other embodiments, the polymer composition may be prepared as a film that may rest on or be supported by a surface or mold during curing, and can then be peeled off as a freestanding film after being cured. The polymer composition can be applied to the surface in an amount such that the freestanding film ranges from about 100 nm to about 300 mm, depending on the size and area of the surface to be coated.

Any substrate or surface useful for the intended application may be used. Substrates may include, by way of example only, flasks, dishes, flat plates, well plates, bottles, containers, pipettes, tubes, membranes, cell culture dishes, and slides. In various embodiments, the substrate can be comprised of any type of material that is suitable for receiving the polymer composition coating. Ideally, although not required, the substrate material will also be conducive to optimal cell growth conditions. Such materials include, but are not limited to, polymeric substrates comprised of glass, polystyrenes, polyacrylates, polyanhydrides, polyurethanes, polyesters, nylons or mixtures thereof, such as those disclosed in U.S. Pat. No. 7,579,179. In one exemplary embodiment, the substrate is a polystyrene well plate.

In various embodiments, one or more surfaces of the substrate to be coated can have any shape. By way of example only, one or more surfaces of the substrate may be flat, curved, or tubular, or have small features. Further, any surface of a substrate may be coated.

In certain embodiments, the monomer/oligomer mixture can be prepared and stored as a monomer or oligomer precursor solution. In various other exemplary embodiments, the polymer composition can be prepared and applied to a substrate and cured, and the substrate stored for future use, in the form of a coated substrate.

In various embodiments, the coating compositions may be formed as a freestanding film. For example, the polymer composition may be applied to a support, and after curing, the cured polymer composition may be removed from the substrate and used, for example as base material for topology-based three-dimensional cell culture products.

Further embodiments relate to methods of culturing cells using the polymer compositions made in accordance with various embodiments of the disclosure. Such methods may comprise, for example, preparing a monomer/oligomer mixture comprising at least one silicone monomer or oligomer and at least one non-silicone monomer or oligomer, adding at least one crosslinker and at least one polymerization initiator to the monomer/oligomer mixture to form a polymer composition, curing the polymer composition by means of an actinic radiation source to form a cured polymer composition; and applying cells to be cultured to the cured polymer composition. In the methods of culturing cells, the polymer composition may be applied to a substrate before curing, or may be formed as a freestanding film.

Any biological application that uses the polymer mixture on a surface, a substrate, or as a freestanding film is within the scope of the disclosure, such as, for example, drug discovery. As a further example, any known cell type may be attached and grown on the substrates coated according to various embodiments described herein. Examples of cell types that can be used include, but are not limited to, nerve cells, epithelial cells, stem cells, fibroblast cells, hepatocytes, breast cells, and other cell types.

For example, liver cell function is of particular interest in the pharmaceutical industry. Drug-induced liver toxicity and unpredicted metabolism are the major causes for drug failures. The primary human hepatocyte model is well accepted in early stage drug screening. However, the long-term metabolic activity of primary hepatocytes is difficult to maintain on rigid plastic dishes that do not permit gas exchange. Recent studies have shown that the CYP450 enzyme activity of primary human hepatocytes is maintained and prolonged in an oxygen rich microenvironment. This study provides a guideline for the design of materials for advanced cell culture substrates, such as materials that allow rapid oxygen exchange. In addition to liver cells, these materials will have the potential to support any highly metabolic cells such as cardiomyocytes, neuronal cells, beta cells, and possibly stem cells when differentiating into a lineage of high metabolism.

Still in further embodiments, the polymers made in accordance with various embodiments of the disclosure can serve as a scaffold in tissue engineering applications. With the aging population, there is an ever-increasing demand for the replacement of degenerative tissues and organs. However, organ donors are limited. Tissue engineering offers a promising solution to restore the lost tissue function. In tissue engineering, scaffold materials provide physical support as well as biochemical cues for the cells. In general, materials that support cells well in vitro are good candidates for in vivo applications, such as implants and engineered tissues. The polymer disclosed herein provides the necessary foundation for advancing tissue engineering materials, particularly for liver, cardiovascular, neuronal and pancreatic tissue regeneration.

In further embodiments, the materials are soft over a range of deniers and transparent. The silicone monomer or oligomer exhibits excellent oxygen permeability for highly metabolic cells. The non-silicone monomer or oligomer, such as a hydrophilic monomer or oligomer, can significantly reduce the uptake of hydrophobic drug molecules by the substrate so that drug-testing experiments can be performed. The hydrophobicity/hydrophilicity of the polymer can be adjusted to suit different cell types. In addition, the polymerization can be initiated using wavelength-specific, non-heat emitting actinic radiation source, such as LED lamps in the visible and near visible range.

Although the disclosure recites components being added or mixed in a particular order, this should not be construed as a requirement that the order is adhered to. It is intended that the components of the polymer composition may be added in any order prior to curing. Ideally, although not required, the composition will be mixed to a desired degree of homogeneity, as would be appreciated by those of skill in the art. Thus, for example, a method of making a polymer composition as disclosed herein in the order comprising mixing at least one silicone monomer or oligomer with at least one non-silicone monomer or oligomer to form a monomer/oligomer mixture; adding at least one crosslinker to the monomer/oligomer mixture; and adding at least one polymerization initiator to the monomer/oligomer mixture, may be performed by combining the recited components in any order prior to curing, and still be within the scope of the disclosure.

Unless otherwise indicated, all numbers used in the specification and claims are to be understood as being modified in all instances by the term “about,” whether or not so stated. It should also be understood that the precise numerical values used in the specification and claims form additional embodiments of the invention. Efforts have been made to ensure the accuracy of the numerical values disclosed in the Examples. Any measured numerical value, however, can inherently contain certain errors resulting from the standard deviation found in its respective measuring technique.

As used herein the use of “the,” “a,” or “an” means “at least one,” and should not be limited to “only one” unless explicitly indicated to the contrary.

It is to be understood that both the foregoing general description and the following detailed description are exemplary and explanatory only and are not restrictive of the invention, as claimed.

The accompanying drawings, which are incorporated in and constitute a part of this specification, are not intended to be restrictive of the invention as claimed, but rather illustrate embodiments of the invention and, together with the description, serve to explain the principles of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.

### EXAMPLES

### Materials and Methods

Exemplary polymer compositions according to various embodiments of the disclosure are set forth in Table 1:

TABLE 1

| Polymer | Silicone | Non-Silicone Monomer | | | | Photo |
|---|---|---|---|---|---|---|
| Type | Monomer | HEMA | VP | DMA | Crosslinker | initiator |
| SH100 | 100 | — | — | — | 3 | 0.3 |
| SH80 | 83 | 17 | — | — | 3 | 0.3 |
| SH67 | 67 | 33 | — | — | 3 | 0.3 |
| SH50 | 50 | 50 | — | — | 3 | 0.3 |
| SH33 | 33 | 67 | | | 3 | 0.3 |
| SH17 | 17 | 83 | | | 3 | 0.3 |
| SV80 | 83 | — | 17 | — | 3 | 0.3 |
| SM80 | 83 | — | — | 17 | 3 | 0.3 |
| SHV80 | 83 | 8.5 | 8.5 | — | 3 | 0.3 |
| SHM80 | 83 | 8.5 | — | 8.5 | 3 | 0.3 |
| SVM80 | 83 | — | 8.5 | 8.5 | 3 | 0.3 |
| SX75 | 75 | 8.5 | 8.5 | 8.5 | 3 | 0.3 |
| SVM75 | 75 | — | 12.5 | 12.5 | 3 | 0.3 |

Table 1 sets forth the chemical compositions of the polymer compositions (ratio by weight) used in the examples. A non-silicone monomer, such as a hydrophilic monomer, for example, HEMA, DMA, or VP, was present in a ratio that ranged from 0% to 83%. A silicone monomer, such as SIM15 or SIM0, was mixed with a non-silicone monomer, such as a hydrophilic monomer, in a polypropylene tube. A crosslinker, such as SIB or R31, was added to the monomer mixture in a ratio of 3% by weight of crosslinker to monomer mixture. SIB is a shorter crosslinker than R31, so that the polymers cross-linked with SIB were more rigid than those with R31. A photo initiator, such as Irgacure 819, was dissolved in an alcohol, such as 200 proof ethanol at a concentration of 2%, before being added to the monomer mixture solution. The photo initiator was present in an amount of about 0.3% to 0.5% of the monomer mixture by weight.

The monomer mixture, crosslinker, and photoinitiator were thoroughly mixed to form a polymer composition, which was applied sequentially to a 96 well polystyrene well plate. A 1 μl to 5 μl drop of the polymer composition was placed in the center of each well of a 96 well plate in this example and allowed to spread and cover entirely each bottom of the individual wells. The plate was then placed in a chemical hood for one hour at room temperature to ensure the mixture completely spread over the bottom of the well, at which time the ethanol also evaporated. The coated plate was purged with N₂ gas in a curing box fitted with a fixed silica window larger than the plate, for 3 minutes before being cured with 375 nm LED lamps, (“UV Cure-All With Lens” lamps manufactured by UV Process Supply, Inc.) at room temperature from one minute to 30 minutes. After curing, the plates were examined for heat damage and warp. There were no unreacted monomers of SH100, SH80 and SH67 detected by HPLC while a minimal amount (<0.02%) of monomers was detected by HPLC for the other mixtures.

No apparent physical changes in terms of heating were found. The coating from all compositions tested had good attachment to the polystyrene (PS) surface and were found to be water insoluble.

The cured polymer compositions made with SIM15 and HEMA, SIM15 and DMA, and SIM15 and VP, all appeared transparent to the unaided eye. These coatings had excellent optical clarity, which is one of the key requirements for cell culture. However, the cured polymer compositions comprising SIM0 and one of the three hydrophilic monomers, HEMA, DMA and VP, were turbid in appearance. Therefore, SIM15 was used for the rest of the experiments.

The stored monomer precursor solutions were stable for at least three weeks when subsequently tested again. The thickness of the coatings is estimated to be 30 μm and 150 μm for 1 μl and 5 μl of monomer precursor solution in 96 well plates, respectively. The coated wells were filled with phosphate buffered saline (PBS) and soaked for up to a two-week period. They were also tested in cell culture conditions for up to a three-week period. In either case, de-lamination of the film from the polystyrene substrate was not observed. The coated plates do not need to be washed with ethanol or PBS. They were UV sterilized with low pressure mercury discharge lamps in a laminar hood for 1 hour before use.

It was noted that the chemical compositions and properties of the cured polymer compositions were controllable and consistent in that they presented minimal batch-to-batch variation.

### Example 1

### Contact Angle

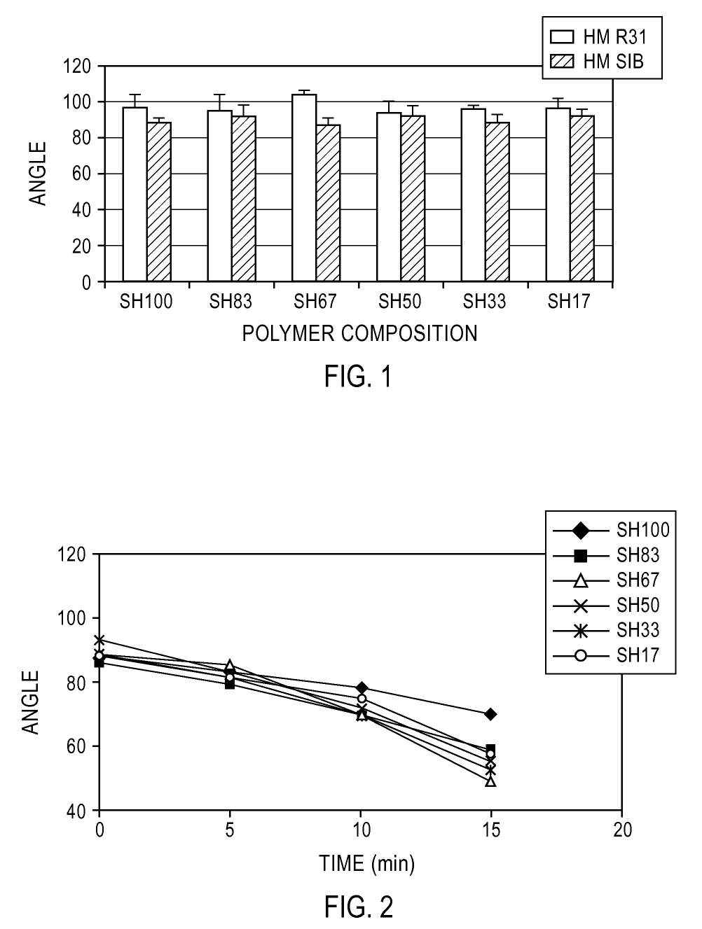

This example demonstrates, by means of a graphical representation (FIG. 1), that the water contact angle of the cured polymer composition (SIM15 and HEMA) was both dynamic and reduced with respect to PDMS.

The polymer compositions used in this example are set forth in Table 1. The polymer composition samples were prepared on glass slides in order to obtain an accurate contact angle measurement. Briefly, a 20 μl drop of the polymer composition was placed on the glass surface and allowed to spread to about a 1 cm² area. The polymer composition was dried and cured as described above. The contact angle of the cured polymer composition containing the hydrophilic monomer HEMA, was between 90° and 100°, regardless of the composition variations when measured immediately after placing the drop of water on the sample, as shown in FIG. 1. Overall, the water contact angle numbers were lower than that of PDMS at ˜110°. Similar results were obtained with the cured polymer compositions when combined with the hydrophilic monomers, VP and DMA when measured immediately after placing the drop on the sample.

FIG. 2 provides a graphical representation of the change in the contact angle of a cured polymer composition comprising SIM15, HEMA, and SIB, as a function of time. The contact angle decreases markedly with time for all the polymers. The more hydrophilic monomers present in the polymer, the more significant a change was observed. Materials with a contact angle of 60° to 80° are desirable for many cell culture applications. These experiments demonstrate the ability to alter the polymer compositions (SH100, SH83, SH67 and SH50) in order to obtain a water contact angle in a desired range.

### Example 2

### Curing Time and Thickness

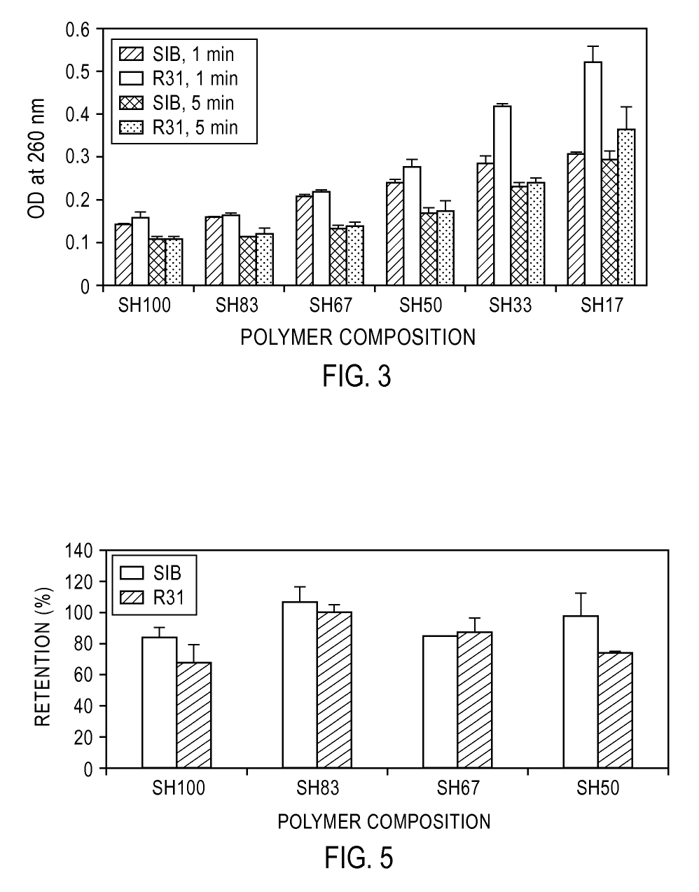

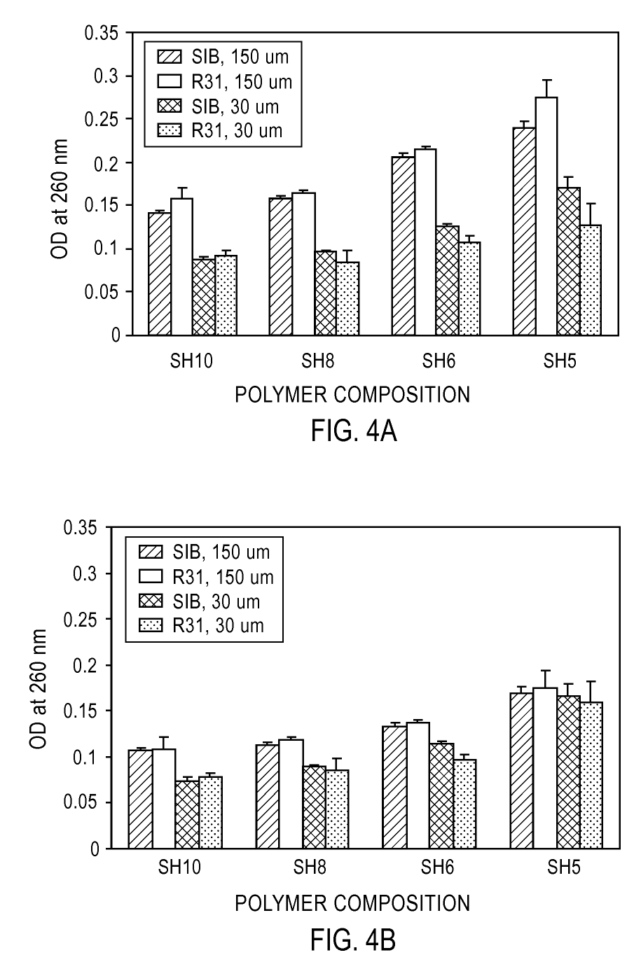

This example demonstrates, by means of a graphical representation (FIGS. 3, 4A and 4B), the effect of curing time at one minute and at five minutes, and coating thickness on unreacted monomer release after exposure to the actinic radiation, including how LED lamps can efficiently cure the polymers and minimize monomer release. Photo-initiated polymerization may generate an incomplete reaction due to various mechanistic reactions known to one of skill in the art. It may be necessary to limit the curing time with conventional broad spectrum lamps due to heating. Heating may trigger undesirable reactions, warp the substrate particularly if made from polystyrene, and degrade biomolecules. As a result, prolonged curing is not always possible and only partial polymerization can be realized. The un-reacted monomers leak out into the cell culture media and some are known to be toxic to cells. With the LED lamps, polymerization can be performed over a long period of time without heating thereby completely curing the formulations and reducing the potential toxicity toward cells. In general, those of skill in the art may recognize the limitations of photo initiators or thermal initiators, for example, and may determine an appropriate method of photo initiation and cure time for the corresponding initiators.

In order to test when the curing is complete using the LED lamps, PBS solution at room temperature was added to the wells of the coated 96 well plates for 24 hours after curing to extract any residual monomers. The solution was then transferred to a 384 well plate and the absorption of the solution at 260 nm was measured using a spectral reader. The optical density (OD) of the control PBS solution under these conditions was found to be ˜0.1. The effect of curing time on the degree of polymerization of the thick (5 μl) polymer composition coatings comprised of SIM15 and HEMA is shown in FIG. 3. A longer cure time of 5 minutes reduced the amount of unreacted monomers with either of the crosslinkers, SIB or R31. After 5 minutes of curing, SH100, SH83, and SH67 did not release any monomers, suggesting they were completely cured.

The thickness of the coating influences the curing efficiency based on how well the light penetrates the film. Less time was required to cure the polymer compositions for a thin coating using 1 μl of the polymer composition comprising SIM15 and HEMA. The monomer release for a thin coating with 1 minute curing time is shown in FIG. 4A. As can be seen, 1 minute was sufficient cure time for a thin layer of SH100, SH83 and SH67 with either crosslinker, SIB and R31.

Therefore, there is a potential to cure a broad range of coating thickness with LED lamps as the lamps emit no radiant heat and can be used continuously for hours without changing output intensity. Where a thick coating is preferred for cell culture, LED lamps may be capable of curing the polymer composition with a minimum un-reacted monomers left behind. In addition, peptides and proteins do not adsorb the wavelengths of the LED lamps used, so they will not be cross-linked or denatured under prolonged exposure.

### Example 3

### Drug Absorption

This example demonstrates by means of a graphical representation (FIG. 5) that absorption of nefazodone was reduced according to various embodiments of the disclosure. Drug absorption studies were conducted by incubating the cured polymer composition coatings with a 200 μM nefazodone solution and measuring the concentration of the drug in solution after 24 hours. The results for nefazodone absorption by the silicone and HEMA polymer compositions are shown in FIG. 5. For each composition cross-linked with SIB, the retention of the drug remaining in solution was more than 85%; with R31, the retention was higher than 70%; and some compositions such as SH83 had retention close to 100%.

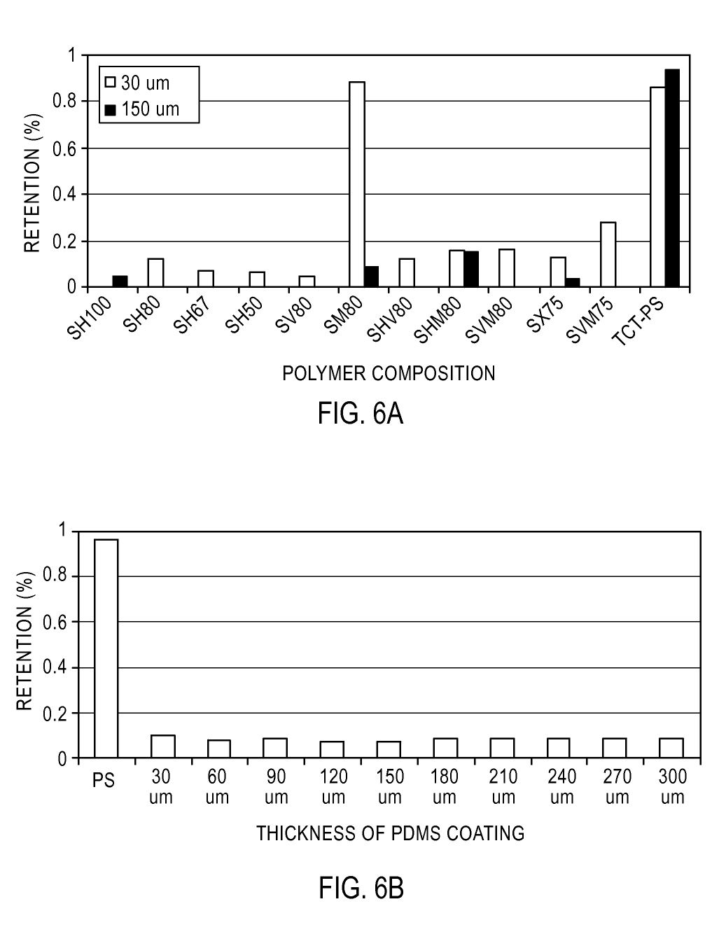

This example further demonstrates by means of graphical representations (FIGS. 6A and 6B) comparing the uptake of Tamoxifen, an exemplary hydrophobic drug, by the different polymer compositions at varying coating thickness prepared in accordance with exemplary embodiments of the present disclosure (FIG. 6A) and the absorption of Tamoxifen by PDMS at varying thickness (FIG. 6B). The coated 96 well plates were sterilized using the low pressure mercury discharge lamps in a laminar flow tissue culture hood for 1 hour before seeding cells. The percent of remaining Tamoxifen after absorption is plotted.

### Example 4

### Breast Cell Culture

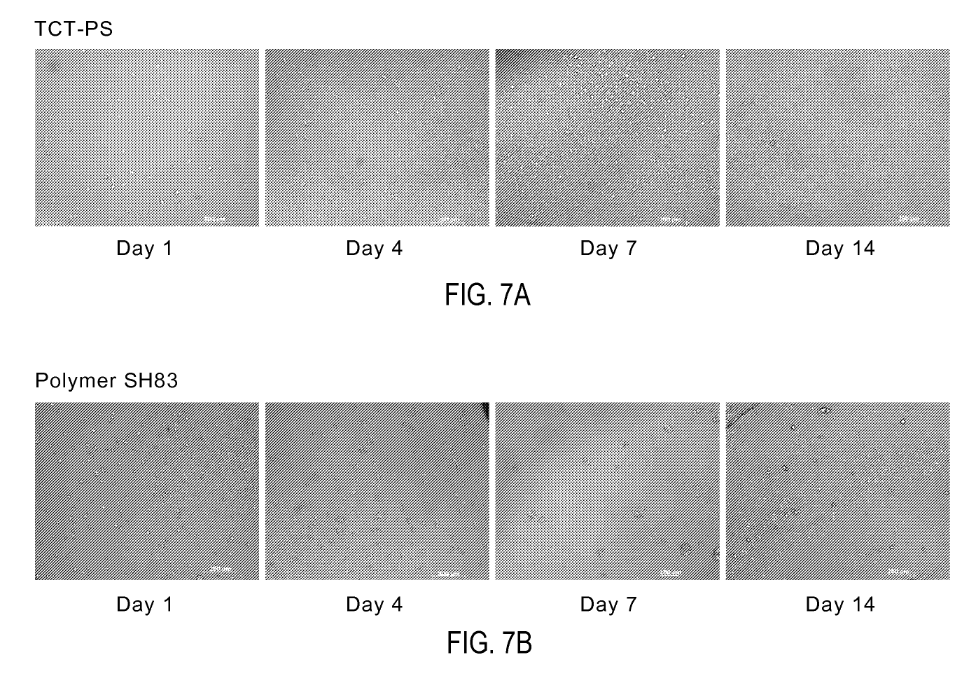

This example demonstrates by means of micrograph representations (FIGS. 7A and 7B) the different cell growth patterns on two cell culture substrates, FIG. 7A, Tissue Culture Treated polystyrene (TCT-PS) substrate, a commercially available substrate, and FIG. 7B, the substrate coated with an exemplary polymer composition according to the present disclosure. The coated 96 well plates were UV sterilized using low pressure mercury discharge lamps (different than those UV LED lamps) in a laminar flow hood for 1 hour followed by rinsing with phosphate buffered saline (PBS) three times before seeding. Breast cells MCF-10A (commercially available cells of a non-tumorgenic epithelial cell line) were seeded at a density of 10,000 cells per well and maintained in a 5% CO₂ incubator at 37° C. The media was refreshed every other day.

Substrate coatings made from the polymer compositions with HEMA, with DMA, and with VP were used in the example. The cells showed different morphology on the different polymer composition coatings. Polystyrene (PS) is a hard material and the cells may respond to the hardness of TCT-PS. In FIG. 7B, the morphology of cells on SH83 (SIM15/HEMA) was compared to that seen on TCT-PS in FIG. 7A, over a period of 2 weeks. A day after seeding, most cells on the TCT-PS spread and attached to the surface. Most cells on SH83, however, remained round-shaped without spreading, which is the first step required for the cells to form acini structures. On day 4, the cells were well attached to the surface and proliferated to about 95% confluence on TCT-PS. In comparison, on day 4, the cells started to show a cluster structure on SH83. On day 7, the cells became confluent and no clusters were observed on TCT-PS. On SH83, the cells formed round-shaped aggregates and the size was bigger than the initial clusters, indicative of the early stage of acini formation. On day 14, the cells were over crowded on TCT-PS with no acini formation. Fewer cells were seen on the SH83 surface, possibly due to the aggregates being loosely attached to the surface and easily removed during medium change. This is very similar to what is seen when MCF-10A cells are cultured on flat PDMS. Nevertheless, the preliminary study demonstrates that the cured polymer compositions according to various embodiments described herein have the physiochemical properties necessary to facilitate potential breast cell acini formation. Moreover, the chemical composition of the polymer composition disclosed herein can be adjusted to accommodate the culture of different types of cells, such as hepatocytes and stem cells.

### Example 5

### Toxicity and Retention Study with C3A Cells

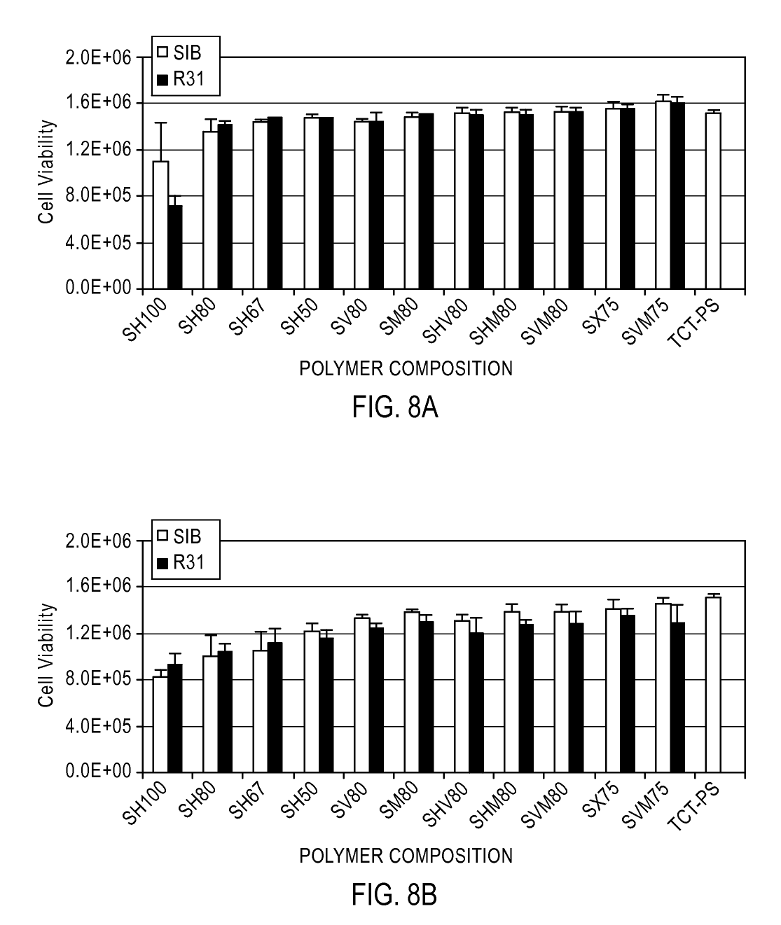

This example demonstrates by means of a graphical representation (FIGS. 8A and 8B) a toxicity and retention study with the C3A cells, HepG2/C3A, in cell culture. HepG2/C3A cells (C3A) were cultured under standard conditions in EMEM (Eagle's minimal essential medium for maintaining cells in tissue culture) containing 10% FBS and 1% PeniStrep. Prior to seeding in cell culture microplates, the C3A cells grown in tissue culture flasks were dislodged by trypsin digestion and then re-suspended in fresh EMEM. Cell density was measured with a Coulter particle counter (Coulter Corporation) before being seeded at a density of 40,000/well in 96-well collagen I or Matrigel™ coated TCT-PS plates.

Culture media was added to the coated plate and incubated overnight in order to differentiate toxicity due to unreacted monomers that leaked into the media as opposed to the cross-linked polymeric material surfaces during culture. The conditioned media of polymer compositions made according to embodiments of the disclosure was then added to the C3A cells that had been seeded on a TCT-PS plate 24 hrs earlier. The viability of C3A cells was determined by comparing the viable cell counts on the new surfaces to those on TCT-PS. Cell viability was measured using the standard colorimetric MTS assay, for assessing the viability (cell counting) and the proliferation of cells (cell culture assays). As can be seen in FIG. 8A, there was no significant toxicity from the conditioned media except for SHI00. FIG. 8B shows the viability of the cells on the silicone acrylate with HEMA polymers as 60-80% of that on the TCT-PS, while the viability on the silicone acrylate with DMA or VP polymer was about 95% of that on the TCT-PS. The results indicate the excellent compatibility of these new materials with hepatocytes and the potential to optimize the material compositions for better cell viability.

### Example 7

### Viability and Cytochrome P450 3A4 Enzymatic Assay with PHH

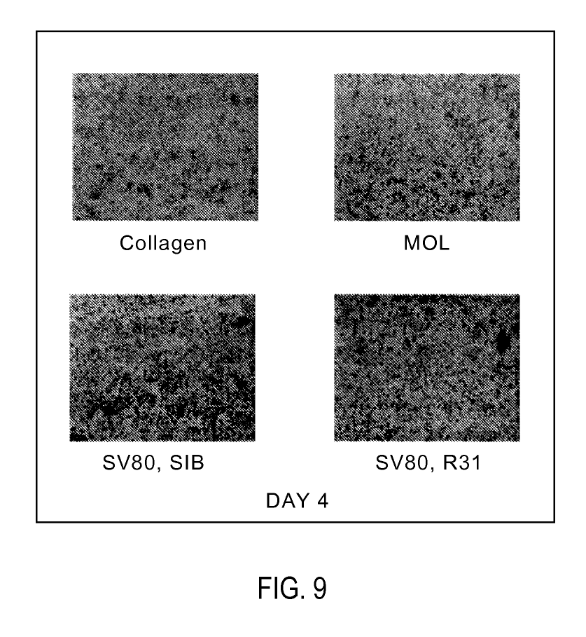

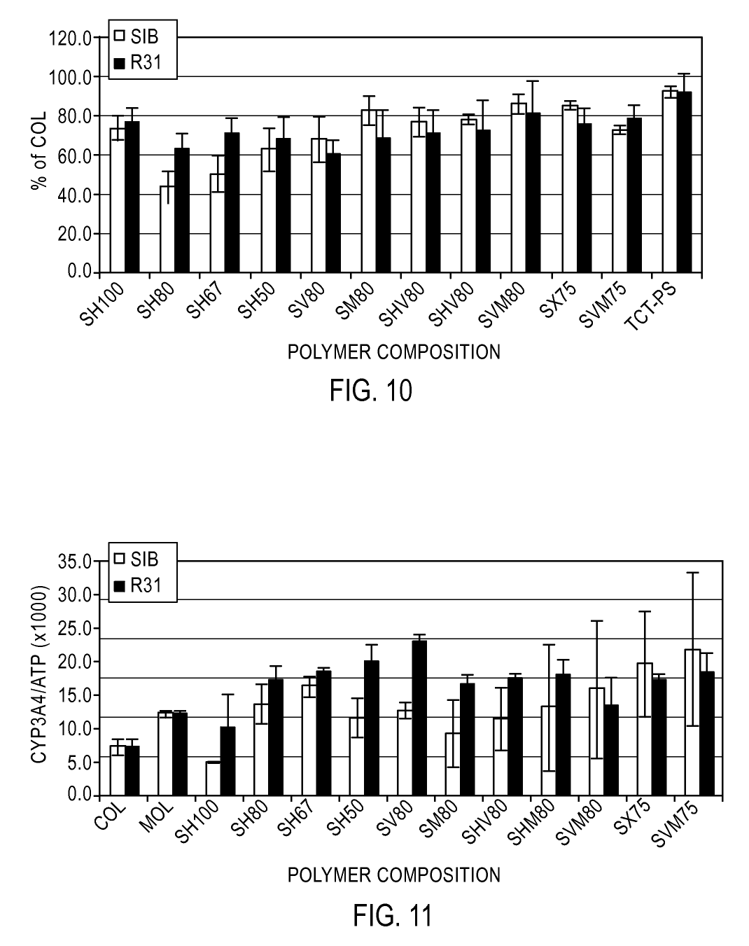

This example demonstrates by way of a photomicrograph representation comparing primary human hepatocytes (PHH) on collagen, Matrigel™ overlay (MOL) and synthetic substrates (FIG. 9) and a graphical representation of the retention of PHH on various substrates according to various embodiments of the disclosure (FIG. 10). Cryopreserved PHH were thawed and purified using a Percoll isolation kit according to the manufacturer's protocol. The purified PHH were then re-suspended in MFE™-p and plated at a density of 60,000/well in 96-well assay plates. The PHH were allowed to attach to the surface by incubating overnight at 37° C., 95% humidity and 5% CO₂. After the PHH attached to the surface, MFE™-p was replaced with MFE™-m. MOL was performed 18-24 hours after seeding. The final concentration of overlaid Matrigel™ was 0.25 mg/ml. The medium was refreshed with new MFE™-m every 48 hours.

FIG. 9 shows the morphology of the primary cells on various substrates observed using optical microscopy. For purposes of simplicity, only the cells on collagen, MOL, SV80 (SIB) and SV80 (R31) were compared. As seen in FIG. 9, there is no apparent difference in the appearance of the cells on the claimed polymers, MOL and collagen. Similar cell morphology was observed on other claimed polymer surfaces. The morphological observation suggests that the new materials are compatible with the primary cells.

After 7 days, the number of viable cells on cured polymer compositions made in accordance with embodiments of the disclosure was compared to the number of viable cells on collagen (FIG. 10). The viability on most polymer materials was about 80%, as compared to 90% on TCT-PS. The effect of different chemical compositions on the viability is not apparent.

This example further demonstrates by means of graphical representation the CPY3A4 activity of PHH on various substrates according to various embodiments of this disclosure (FIG. 11). The enzymatic activity of Cytochrome P450 subtype 3A4 was measured using a standard luminescent assay method. PHH cultured in 96-well plates were incubated with 60 ul of MFE™-m containing a 1:1,000 dilution of a 3A4 specific luminogenic CYP450 substrate (Luciferin-IP A). After one hour of incubation, 50 ul of the reaction medium was mixed with 50 ul of P450-Glo™ Luciferin Detection Reagent. After 15 minutes of incubation, the luminescent intensity was measured using a Victor III luminometer (Perkin-Elmer). The ATP levels of the cells were measured using a CellTiter-GLO™ ATP assay kit (Promega) and used for cell number normalization.

Compared to MOL, the CPY3A4 enzyme activity was increased on the polymer surfaces except for SH100. The highest activity (2 times that of MOL) was on the SV surface which contained VP and the long crosslinker in the composition (FIG. 11). The results confirm that the claimed polymers can support liver cell function and can be used as a cell culture substrate and tissue engineering scaffold.

### Example 8

This example describes how a cured polymer composition made according to an embodiment of the disclosure is moldable into different topologies, and in particular how it can be formed as a freestanding film. 50 μl of the monomer mixture was placed on top of a 1 cm² micro-patterned PDMS mold. After the ethanol evaporated, the monomer mixture was cured with LED lamps for 10 minutes. The film was easily peeled off the PDMS mold, and the topology from the PDMS was faithfully transferred to the cured polymer composition. Therefore, the new materials can serve as the foundation material for topology-based 3D cell culture products to retain cell aggregates.

What is claimed is:

1. A method of coating a cell culture substrate, said method comprising:
preparing a monomer/oligomer mixture comprising:
at least one silicone monomer or oligomer and at least one non-silicone monomer or oligomer;
at least one crosslinker; and
at least one polymerization initiator;
applying the monomer/oligomer mixture to the cell culture substrate; and curing the monomer/oligomer mixture with a non-heat emitting actinic radiation source to form a polymer composition.

2. The method according to claim 1, wherein the at least one silicone monomer or oligomer contains at least one (meth)acrylate moiety.

3. The method according to claim 2, wherein the at least one (meth)acrylate moiety is chosen from (meth)acryloxyethoxytrimethylsilane and (3-(meth)acryloxy-2-hydroxypropoxy)propylbis(trimethylsiloxy)methylsilane.

4. The method according to claim 1, wherein the at least one non-silicone monomer or oligomer is a hydrophilic monomer or oligomer chosen from 2-hydroxyethyl(meth)acrylate, N,N-di(meth)lacrylamide, 1-vinyl-2-pyrrolidinone, and carboxyethyl acrylate.

5. The method according to claim 1, wherein the at least one non-silicone monomer or oligomer is present in an amount ranging from about 0% to about 83% by weight.

6. The method according to claim 1, wherein the actinic radiation source is a non-heat emitting UV lamp.

7. The method according to claim 1, wherein the actinic radiation source is a non-heat emitting LED lamp.

8. A method of preparing a polymer composition as a freestanding film suitable for cell culture applications, said method comprising:
preparing a monomer/oligomer mixture comprising:
at least one silicone monomer or oligomer and at least one non-silicone monomer or oligomer;
at least one crosslinker; and
at least one polymerization initiator;
applying the monomer/oligomer mixture to a support;
curing the monomer/oligomer mixture with a non-heat emitting actinic radiation source to form a polymer composition; and
removing the cured polymer composition from the support to form a freestanding film suitable for cell culture applications.

9. The method according to claim 8, wherein the at least one silicone monomer or oligomer contains at least one (meth)acrylate moiety.

10. The method according to claim 9, wherein the at least one (meth)acrylate moiety is chosen from (meth)acryloxyethoxytrimethylsilane and (3-(meth)acryloxy-2-hydroxypropoxy)propylbis(trimethylsiloxy)methylsilane.

11. The method according to claim 8, wherein the at least one non-silicone monomer or oligomer is a hydrophilic monomer or oligomer chosen from 2-hydroxyethyl(meth)acrylate, N,N-di(meth)lacrylamide, 1-vinyl-2-pyrrolidinone, and carboxyethyl acrylate.

12. The method according to claim 8, wherein the at least one non-silicone monomer or oligomer is present in an amount ranging from about 0% to about 83% by weight.

13. The method according to claim 8, wherein the actinic radiation source is a non-heat emitting UV lamp.

14. The method according to claim 8, the actinic radiation source is a non-heat emitting LED lamp.

15. A method of culturing cells, said method comprising:
preparing a monomer/oligomer mixture comprising at least one silicone monomer or oligomer and at least one non-silicone monomer or oligomer;
adding at least one crosslinker and at least one polymerization initiator to the monomer/oligomer mixture to form a polymer composition;
curing the polymer composition by means of an actinic radiation source to form a cured polymer composition; and
applying cells to be cultured to the cured polymer composition.

16. The method according to claim 15, wherein the at least one silicone monomer or oligomer contains at least one (meth)acrylate moiety.

17. The method according to claim 16, wherein the at least one (meth)acrylate moiety is chosen from (meth)acryloxyethoxytrimethylsilane and (3-(meth)acryloxy-2-hydroxypropoxy)propylbis(trimethylsiloxy)methylsilane.

18. The method according to claim 15, wherein the at least one non-silicone monomer or oligomer is a hydrophilic monomer or oligomer chosen from 2-hydroxyethyl(meth)acrylate, N,N-di(meth)lacrylamide, 1-vinyl-2-pyrrolidinone, and carboxyethyl acrylate.

19. The method according to claim 15, wherein the at least one non-silicone monomer or oligomer is present in an amount ranging from about 0% to about 83% by weight.

20. The method according to claim 15, wherein the actinic radiation source is a non-heat emitting UV lamp.

21. The method according to claim 15, wherein the actinic radiation source is a non-heat emitting LED lamp.

22. The method according to claim 15, wherein the polymer composition is applied to a substrate before it is cured.

23. The method according to claim 15, wherein the polymer composition is formed as a freestanding film.
